(19) United States
(12) Patent Application Publication
(10) Pub. No.: US 2024/0115170 A1
(43) Pub. Date: Apr. 11, 2024

(54) TWO-STAGE BLOOD GLUCOSE PREDICTION METHOD BASED ON PRE-TRAINING AND DATA DECOMPOSITION

(71) Applicants: Shaoda ZHANG, Guangdong (CN); Zheng WANG, Guangdong (CN); Xingyu ZHENG, Guangdong (CN)

(72) Inventors: Shaoda ZHANG, Guangdong (CN); Zheng WANG, Guangdong (CN); Xingyu ZHENG, Guangdong (CN)

(21) Appl. No.: 18/206,095

(22) Filed: Jun. 6, 2023

(30) Foreign Application Priority Data
Sep. 22, 2022 (CN) 202211157514.9

(51) Int. Cl. A61B 5/145; A61B 5/00; G16H 50/70

(52) U.S. Cl. CPC A61B 5/14532; A61B 5/7246; A61B 5/725; A61B 5/7267; G16H 50/70

(57) ABSTRACT

The present disclosure relates to a two-stage blood glucose prediction method based on pre-training and data decomposition. The method includes the following steps: combining blood glucose data of healthy people and diabetic people to develop a pre-training model; collecting data of diabetic patients to be predicted; performing missing value imputation processing and smooth processing on the data of the diabetic patients; performing mode decomposition on the data; performing sample entropy analysis; importing the processed data of the diabetic patients into an ensemble learning module. In accordance with the present disclosure, the blood glucose data of healthy people and diabetic people are combined at first to train a blood glucose prediction model as a pre-training model, and the model is enabled to have a prediction data reserve, so as to solve the problem that a blood glucose concentration of a patient outside samples cannot be predicted well.

### BACKGROUND OF THE INVENTION

The present disclosure relates to the field of blood glucose prediction, and in particular to a two-stage blood glucose prediction method based on pre-training and data decomposition.

Diabetes is a metabolic disease caused by the disruption of insulin secretion. The glucose in the body of patient cannot be absorbed normally, which will lead to short-term or long-term complications in the long run, seriously affecting the quality of life and life safety of the patient. Blood glucose concentration is the standard for diagnosing diabetes. Continuous blood glucose data collection from patients is obtained with the help of CGMS (Continuous Glucose Monitoring system), and then blood glucose prediction can be performed.

One of the common methods of blood glucose prediction is based on a data-driven model, which only considers the blood glucose data of a patient, and the blood glucose concentration change in the future is predicted by using the recent blood glucose value and combining an algorithm, such as, a recursive neural network proposed by Sandham, an autoregressive model proposed by Bremer, an approach employing a self-feedback neural network proposed by Fayrouz, the use of a support vector machine algorithm proposed by Georga, the use of extreme learning machine algorithm proposed by Mo Xue et al., and a chaotic prediction model of blood glucose established by Li Ning et al. using echo state networks. The blood glucose prediction model can be established by using above algorithms, the algorithms can be verified by patient data, thus obtaining more accurate experimental results. This method uses only the historical blood glucose data of patients for blood glucose prediction, without considering other physiological factors.

At present, only one of the above models is used for blood glucose prediction, and the blood glucose concentration of patients outside the sample data cannot be predicted well as single methods are poor generalization ability and mostly consider only the blood glucose data of a single diabetic patient.

The above problems are worth addressing.

### BRIEF SUMMARY OF THE INVENTION

In order to overcome the problems in the prior art that the blood glucose concentration of patients outside the sample data cannot be predicted well due to the fact that only one model, which only considers the blood glucose data of a single diabetic patient and is poor in generalization ability, is used for blood glucose prediction, a two-stage blood glucose prediction method based on pre-training and data decomposition is provided in accordance with the present disclosure.

The technical solution provided by the present disclosure is as follows:

A two-stage blood glucose prediction method based on pre-training and data decomposition includes the following steps:
S1, combining blood glucose data of healthy people and diabetic people to develop a pre-training model;
S2, collecting data of diabetic patients to be predicted;
S3, performing missing value imputation processing and smooth processing on the data obtained in S2;
S4, performing mode decomposition on the data obtained in S3 to decompose the data into intrinsic mode components with different frequency information;
S5, performing sample entropy analysis on the mode components obtained by decomposing in S4, and performing secondary decomposition on a component with the maximum sample entropy; and
S6, loading a weight of the pre-training model obtained in S1, and importing the data of the diabetic patients processed in Step 5 into an ensemble learning module, wherein the ensemble learning module is used for predicting blood glucose values in the next 30 minutes and the next 60 minutes.

According to the present disclosure with the above solution, S1 includes the following steps.

S101. A first database is imported, and samples in the first database include the blood glucose data of the diabetic people and the blood glucose data of the healthy people.

The data in the first database includes low blood glucose index (LBGI) and high blood glucose index (HBGI).

An algorithm for the LBGI is to statistically transform blood glucose monitoring results, calculate the low blood glucose index according to the transformation results, and then calculate the average of all low blood glucose index values; the formula is as follows:

$$L B G I = \frac{1}{n} \sum ( 10 \times {( fbgi < 0 )}^{2} ) .$$

An algorithm for the HBGI is to statistically transform blood glucose monitoring results, calculate the high blood glucose index according to the transformation results, and then calculate the average of all high blood glucose index values; the formula is as follows:

$$H B G I = \frac{1}{n} \sum ( 10 \times {( fbgi > 0 )}^{2} ) ;$$

$$fbgi = 1.509 \times ( {log ( B G )}^{1.084} - 5.381 ) ;$$

in the formula, fbgi is a transformed blood glucose value, and n is the total number of blood glucose measurements.

The sum of the low blood glucose index and the high blood glucose index is taken as Risk Index, i.e.,

Risk Index=LBGI+HBGI.

S102. Historical blood glucose data of the past 30 minutes, the past 1 hour, the past 2 hours, the past 4 hours and the past 8 hours are screened out.

S103. The screened blood glucose data are sent to an LSTM (Long-Short Term Memory) model, a training result is saved as a weight file, which is used as a pre-training model and as default parameters of a subsequent training model.

The LSTM mainly achieves a function of information transmission through three gates, e.g., a forget gate, an input gate and an output gate. The forget gate is used to determine to forget how much cell information of the last round of memory via a sigmoid unit through a previous hidden layer and the current input layer, i.e., fₜ=σ(W_{xf}xₜ+W_{hf}hₜ₋₁+b_{f}).

The input gate has a sigmoid unit to determine input information and an output ratio, i.e., iₜ=σ(xₜWₓᵢ+hₜ₋₁Wₕᵢ+bᵢ).

The input information is obtained through a tanh unit, i.e., Ĉₜ=tanh(xₜW_{xc}+hₜ₋₁W_{hc}+b_{c}).

The real input information after gating is iₜĈₜ, the cell information is jointly determined by the information left over from the last round and the information obtained at present, so cₜ=fₜcₜ₋₁+iₜ*Ĉₜ, i.e.,

cₜ=fₜcₜ₋₁+iₜ*tanh(xₜW_{xc}+hₜ₋₁W_{hc}+b_{c}).

The obtained cell information is processed via the tanH unit to obtain output information of the hidden layer, and at the same time, there is an output gate that controls an output through the sigmoid unit, including:
oₜ=σ(xₜWₓₒ+hₜ₋₁Wₕₒ+cₜ₋₁W_{co}+bₒ);
hₜ=oₜ tanh(cₜ);
a final output ŷₜ is: ŷₜ=softmax (hₜ).

Further, the blood glucose data in S101 is continuous blood glucose monitoring data of 50 consecutive days. Sample population include multiple children, multiple adolescents and multiple adults.

According to the present disclosure with the above solution, S2 includes the following steps.

S201. Historical blood glucose data of the diabetic patient to be predicted are collected as a second database.

S202. The second database is imported.

Further, requirements for blood glucose data collection in S201 includes that a blood glucose testing instrument must collect for at least 4 days in 7 consecutive days, and that at least 96 hours of continuous blood glucose data must be collected, in which at least 24 hours are spent overnight (i.e., from 10 pm to 6 am).

Furthermore, samples of the second database are the blood glucose data of patients with type 1 diabetes, and the age of the sample population ranges from 3.5 to 17.7 years old, with an average age of 9.9 years old.

According to the present disclosure with the above solution, S3 includes the following steps.

S301. Patient blood glucose data including missing values are processed by using a data missing value imputation method.

S302. The blood glucose data is smoothed by using a data smoothing filtering method.

Further, the data missing value imputation method includes bilinear interpolation and linear extrapolation, and the data smoothing filtering method includes Kalman filtering and median filtering.

According to the present disclosure with the above solution, S4 includes the following steps.

S401. Historical blood glucose data of the past 1 hour, the past 3 hours, and the past 8 hours are chosen.

S402. The chosen data is subjected to rolling decomposition by using an ensemble empirical mode decomposition model, a time step of the rolling decomposition is set to be two days, so as to obtain signals with different frequencies, that is, multiple IMF components.

Specific decomposition steps of CEEMDAN (Complete Ensemble Empirical Mode Decomposition with Adaptive Noise) are as follows.

(1) Gaussian white noise is added to a signal y(t) to be decomposed to obtain a new signal y(t)+(−1)^{q}εv^{j}(t), where q=1, 2, the new signal is subjected to EMD (empirical mode decomposition) to obtain a first-order intrinsic mode component C₁:

E(y(t)+(−1)^{q}εv^{j}(t))=C₁^{j}(t)+r^{j}

in which, Eᵢ (·) is an i-th intrinsic mode component obtained after EMD decomposition, v^{j} is a Gaussian white noise signal satisfying a standard normal distribution, j=1, 2, . . . , N is the number of times of adding white noise, E is a standard table of white noise, and y(t) is a signal to be decomposed.

(2) N generated mode components are subjected to overall average to obtain a first intrinsic mode component after CEEMDAN decomposition:

$$\overline{{C}_{1} ( t )} = \frac{1}{N} {\sum }_{J = 1}^{N} {C}_{1}^{j} ( t ) .$$

(3) A residual after removing the first mode component is calculated:

r₁(t)=y (t)−C̅₁̅(̅t̅)̅.

(4) Positive-negative paired Gaussian white noises are added to rᵢ(t) to obtain

a new signal, and the EMD decomposition is performed with the new signal as a carrier to obtain the first-order mode component D₁, thus obtaining a second intrinsic mode component after CEEMDAN decomposition:

$$\overline{{C}_{2} ( t )} = \frac{1}{N} {\sum }_{J = 1}^{N} {D}_{1}^{j} ( t ) .$$

(5) A residual after removing the second mode component is calculated:
r₂(t)=r₁(t)−C̅₂̅(̅t̅)̅.

(6) Above steps are repeated until an obtained residual signal is a monotone function and cannot be decomposed continuously, and the algorithm is end. In a case that the number of the intrinsic mode components obtained at this time is k, the original signal y(t) is decomposed into:

y(t)=Σₖ₌₁^{K} C̅ₖ̅(̅t̅)̅+rₖ(t).

According to the present disclosure with the above solution, S5 includes the following steps.

S501. The degree of chaos among the IMF components is calculated, and calculated entropy values are ranked according to results from large to small.

S502. A component with the maximum entropy value is subjected to secondary decomposition to maintain entropy values of all decomposed components within a certain interval, thus reducing nonlinearity and non-stationarity of the blood glucose data.

Furthermore, a variational mode decomposition model is adopted when performing the secondary composition on the component with the maximum entropy value.

According to the present disclosure with above solution, the ensemble learning module in step S6 includes multiple different machine learning algorithms. Importing the data of the diabetic patients processed in S5 specifically includes the following steps.

S601. The data is sent to three different machine learning algorithms, such as an LSTM, a GRU (Gate Recurrent Unit) and a SRNN (Sliced Recurrent Neural Network), so as to obtain multiple prediction results.

S602. The multiple prediction results are combined as a basic prediction result.

S603. The basic prediction result obtained in step S602 is used as a training set, and the training set is sent to a model Nested-LSTM to obtain a final prediction result.

The present disclosure has the beneficial effects as follows:

In accordance with the present disclosure, the blood glucose data of healthy people and diabetic people are combined at first to train a universal blood glucose prediction model as a pre-training model, and the model is enabled to learn blood glucose characteristics of a batch of diabetic people and healthy people in advance to have a prediction data reserve. Afterwards, for a diabetic patient needing to be predicted, a blood glucose concentration of the diabetic patient in the next 30 minutes and 60 minutes can be predicted by combining relevant blood glucose characteristics and historical blood glucose data of the diabetic patient.

A preliminary blood glucose prediction result is obtained by weighted superposition of model prediction results. Multiple machine learners are constructed and combined to complete a learning task. Different network models can learn and combine the corresponding blood glucose characteristics, so as to achieve a better blood glucose prediction effect.

Further, the patient blood glucose data including missing values is processed to make the collected CGM data more stable and closer to the real blood glucose data.

Further, after using rolling decomposition, the difference of the blood glucose data after each rolling prediction can be well observed.

Further, entropy values of subsequences obtained after rolling decomposition are ranked from large to small by using a sample entropy and a permutation entropy, and the prediction effect of the model can be improved by ranking the ease of prediction of the sub-sequences obtained after each decomposition.

Described above is merely an overview of the inventive scheme. In order to more apparently understand the technical means of the disclosure to implement in accordance with the contents of specification, and to more readily understand above and other objectives, features and advantages of the disclosure, specific embodiments of the disclosure are provided hereinafter.

### BRIEF DESCRIPTION OF THE DRAWINGS

FIG. 1 is a flow chart of a method in accordance with the present disclosure.

FIG. 2 is a graph of a blood glucose concentration curve before bilinear interpolation processing.

FIG. 3 is a graph of blood glucose concentration curve after bilinear interpolation processing.

FIG. 4 is a graph of blood glucose concentration curve before linear extrapolation processing.

FIG. 5 is a graph of blood glucose concentration curve after linear extrapolation processing.

FIG. 6 is an effect diagram of a blood glucose prediction in accordance with the present disclosure.

### DETAILED DESCRIPTION OF THE INVENTION

In order to understand the objectives, technical solutions and technical effect of the present disclosure better, the present disclosure is further explained below in conjunction with the accompanying drawings and embodiments. Meanwhile, it is stated that the embodiments described below are only used to explain the present disclosure rather than limiting the present disclosure.

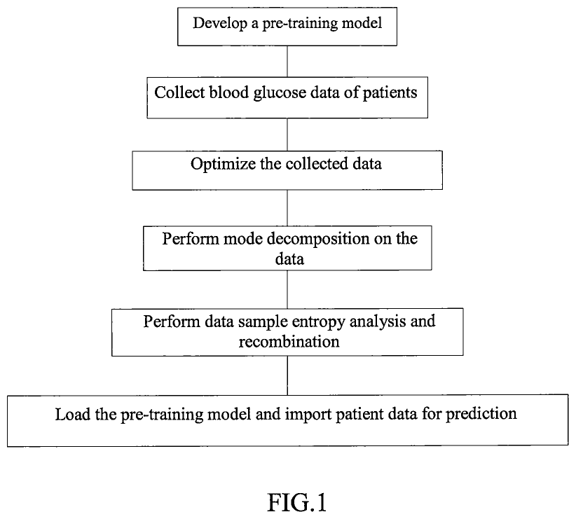

As shown in FIG. 1, a two-stage blood glucose prediction method based on pre-training and data decomposition includes the following steps:

S1, combining blood glucose data of healthy people and diabetic people to develop a pre-training model;

S2, collecting data of diabetic patients to be predicted;

S3, performing missing value imputation processing and smooth processing on the data obtained in S2;

S4, performing mode decomposition on the data obtained in S3 to decompose the data into intrinsic mode components with different frequency information;

S5, performing sample entropy analysis on the mode components obtained by decomposing in S4, and performing secondary decomposition on a component with the maximum sample entropy; and

S6, loading a weight of the pre-training model obtained in S1, and importing the data of the diabetic patients processed in Step 5 into an ensemble learning module, wherein the ensemble learning module is used for predicting blood glucose values in the next 30 minutes and the next 60 minutes.

In the present disclosure, S1 includes the following steps:

S101. A first database is imported, and samples of the first database include the blood glucose data of the diabetic people and the blood glucose data of healthy people. The blood glucose data in S101 is continuous blood glucose monitoring data of 50 consecutive days, and sample population include multiple children, multiple adolescents and multiple adults. Part of data in the first database are shown in the following table:

| | 70 <= BG <= 180 | BG > 180 | BG < 70 | BG > 250 | BG < 50 | LBGI | HBGI | Risk Index |
|---|---|---|---|---|---|---|---|---|
| adolescent#001 | 100 | 0 | 0 | 0 | 0 | 0.09676098 | 0.463578908 | 0.560339888 |
| adolescent#002 | 65.54405944 | 26.24123325 | 8.214707312 | 1.71515867 | 2.159572252 | 1.171796584 | 3.318713869 | 4.490510454 |
| adolescent#003 | 90.22290119 | 7.64530241 | 2.131796403 | 0 | 0.041663773 | 0.450470542 | 1.173219672 | 1.623690214 |
| adolescent#004 | 83.77196028 | 10.02708145 | 6.200958267 | 0 | 0.416637733 | 1.112974384 | 1.427978028 | 2.540952412 |
| adolescent#005 | 72.9046594 | 21.48461912 | 5.610721478 | 2.076244705 | 1.520727727 | 0.803204025 | 1.980683 | 2.783887026 |
| adolescent#006 | 96.99326436 | 3.006735643 | 0 | 0 | 0 | 0 | 0.650280805 | 0.650280805 |
| adolescent#007 | 60.16943268 | 30.09513228 | 9.735435039 | 6.666203736 | 3.374765641 | 1.517738784 | 3.348977457 | 4.866716241 |
| adolescent#008 | 74.29345184 | 23.713631 | 1.992917159 | 4.367752239 | 0.201374905 | 0.200909844 | 3.031484356 | 3.2323942 |
| adolescent#009 | 87.41059649 | 9.811818624 | 2.7778489 | 0 | 0.034719811 | 0.449631403 | 1.241904231 | 1.691535634 |
| adolescent#010 | 100 | 0 | 0 | 0 | 0 | 0.15347298 | 0.550416861 | 0.703889841 |
| adult#001 | 94.75036456 | 2.076244705 | 3.173390737 | 0 | 0.152767169 | 0.952691517 | 0.838390316 | 1.791081834 |
| adult#002 | 99.75001736 | 0 | 0.24998264 | 0 | 0 | 0.746803859 | 0.138988932 | 0.88579279 |
| adult#003 | 99.4861468 | 0.513853205 | 0 | 0 | 0 | 0.000432686 | 1.037869297 | 1.038301983 |
| adult#004 | 91.10478439 | 8.89521561 | 0 | 0 | 0 | 0.036388601 | 0.869165834 | 0.905554436 |
| adult#005 | 99.7986251 | 0.201374905 | 0 | 0 | 0 | 0.081975835 | 0.587434717 | 0.669410552 |
| adult#006 | 97.02104021 | 2.180404139 | 0.798555656 | 0 | 0 | 0.31741661 | 0.997322393 | 1.314739003 |
| adult#007 | 100 | 0 | 0 | 0 | 0 | 0.111917245 | 0.372978494 | 0.484895739 |
| adult#008 | 99.88195264 | 0 | 0.118047358 | 0 | 0 | 0.441736332 | 0.210514029 | 0.652250361 |
| adult#009 | 78.37650163 | 2.972015832 | 18.65148254 | 0 | 5.020484689 | 3.586734245 | 0.783422917 | 4.370157161 |
| adult#010 | 92.73661551 | 7.068953545 | 0.194430942 | 0 | 0 | 0.21420621 | 1.348410878 | 1.562617088 |
| child#001 | 77.23074786 | 8.513297688 | 14.25595445 | 0.138879244 | 0.270814527 | 1.442716999 | 0.085306997 | 1.528023996 |
| child#002 | 77.45989862 | 0.458301507 | 22.08179988 | 0 | 4.555239219 | 3.43699239 | 0.40437962 | 3.84137201 |
| child#003 | 2.673425457 | 0.631900562 | 96.69467398 | 0.020831887 | 96.43080342 | 48.17427454 | 1.342774247 | 49.51704878 |
| child#004 | 35.89334074 | 0 | 64.10665926 | 0 | 35.86556489 | 16.3029539 | 0.00805608 | 16.31100998 |
| child#005 | 60.69717381 | 0 | 39.30282619 | 0 | 15.36004444 | 9.055627983 | 0.007604162 | 9.063232145 |
| child#006 | 70.8631345 | 11.97833484 | 17.15853066 | 0.305534338 | 3.388653566 | 2.023331011 | 0.679482298 | 2.702813309 |
| child#007 | 86.89674328 | 0 | 13.10325672 | 0 | 0.145823207 | 2.51141117 | 0.023325293 | 2.534736464 |
| child#008 | 0.826331505 | 0.458301507 | 98.71536699 | 0.263870565 | 98.6945351 | 20.07692865 | 6.561082585 | 26.63801124 |
| child#009 | 77.30713145 | 4.701062426 | 17.99180612 | 0 | 1.31240886 | 1.821780755 | 0.170821179 | 1.992601935 |
| child#010 | 66.82174849 | 19.90139574 | 13.27685577 | 7.207832789 | 1.999861121 | 1.347517243 | 1.188008525 | 2.535525768 |

A first column indicates a sample category, including 10 adolescents, 10 adults and 10 children; BG is a blood glucose value monitored by CGM. A second column indicates the proportion of blood glucose value in normal blood glucose range (70-180 mg/di). A third column indicates the proportion of blood glucose value in the hyperglycemia range (higher than 180 mg/di). A fourth column indicates the proportion of blood glucose value in the hypoglycemia range (lower than 70 mg/di). A fifth column indicates the proportion of blood glucose value higher than 250 mg/dl. A sixth column indicates the proportion of blood glucose value lower than 50 mg/dl. For example, all blood glucose data of adolescent #010 should be in the range of 70≤BG≤180, providing that this sample belongs to healthy people. For another example, blood glucose data of adult #002 are in the range of 70≤BG≤180 for 99.75% of time and in the range of hypoglycemia for only 0.25% of time, the time of hypoglycemia is less than 4%, providing that the patient is a diabetic patient with better blood glucose control.

In a seventh column in Table, LBGI (Low blood glucose index) is a comprehensive score, which was put forward by Koatchev et al. in 1990s for reflecting the frequency and degree of hypoglycemia events in SMBG in one month and predicting the risk of severe hypoglycemia in the next 3-6 months.

An algorithm for the LBGI is to statistically transform blood glucose monitoring results, calculate the low blood glucose index according to the transformation results, and then calculate the average of all low blood glucose index values. The formula is as follows:

$$L B G I = \frac{1}{n} \sum ( 10 \times {( fbgi < 0 )}^{2} ) .$$

In an eighth column, an algorithm for HBGI (High blood glucose index) is to statistically transform blood glucose monitoring results, calculate the high blood glucose index according to the transformation results, and then calculate the average of all high blood glucose index values. The formula is as follows:

$$H B G I = \frac{1}{n} \sum ( 10 \times {( fbgi > 0 )}^{2} ) ;$$

$$fbgi = 1.509 \times ( {log ( B G )}^{1.084} - 5.381 ) ;$$

in the formula, fbgi is a transformed blood glucose value, and n is the total number of blood glucose measurements.

In a ninth column in Table, Risk Index=LBGI+HBGI.

S1 also includes the following steps.

S102. Historical blood glucose data of the past 30 minutes, the past 1 hour, the past 2 hours, the past 4 hours and the past 8 hours are screened out.

S103. The screened blood glucose data are sent to an LSTM model, and a training result is saved as a weight file, which is used as a pre-training model and as default parameters of a subsequent training model.

In the present disclosure, the LSTM model is a special type of RNN (Recurrent Neural Network) model, which can learn long-term dependent information. The LSTM formula is as follows:

iₜ=σₜ(xₜWₓᵢ+hₜ₋₁Wₕᵢ+bᵢ),

fₜ=σ_{f}(xₜW_{xf}+hₜ₋₁W_{hf}+b_{f}),

cₜ=fₜ⊙cₜ₋₁+iₜ⊙σₜ(xₜW_{xc}+hₜ₋₁W_{hc}+b_{c}),

oₜ=σₒ(xₜWₓₒ+hₜ₋₁Wₕₒ+hₒ),

hₜ=oₜ⊙σₕ(cₜ);

in which σ denotes a logical sigmoid function, iₜ denotes an input gate; fₜ denotes a forget door; cₜ denotes a unit activation vector; o_{f} denotes an output gate; hₜ denotes a hidden layer unit; Wₓᵢ denotes a weight matrix between the input gate and an input feature vector; Wₕᵢ denotes a weight matrix between the input gate and the hidden layer unit; W_{ci} denotes a weight matrix between the input gate and the unit activation vector; W_{xf} denotes a weight matrix between the forget gate and the input feature vector; W_{hf} denotes a weight matrix between the forget gate and the hidden layer unit; W_{cf} denotes a weight matrix between the forget gate and the unit activation vector; Wₓₒ denotes a weight matrix between the output gate and the input feature vector; Wₕₒ denotes a weight matrix between the output gate and the hidden layer unit; W_{co} denotes a weight matrix between the output gate and the unit activation vector; W_{xc} and W_{hc} denote a weight matrix between the unit activation vector and the feature vector and a weight matrix between the unit activation vector and the hidden layer unit, respectively; t denotes the sampling time; tanh is an activation function; bᵢ, b_{f}, b_{c} and bₒ denote deviation values of the input gate, the forget gate, the unit activation vector and the output gate, respectively.

The LSTM mainly achieves the function of information transmission through three gates, e.g., a forget gate, an input gate, and an output gate. The forget gate is used to determine to forget how much cell information of the last round of memory via a sigmoid unit through a previous hidden layer and the current input layer, i.e., fₜ=σ(W_{xf}xₜ+W_{hf}hₜ₋₁+b_{f}).

The input gate has a sigmoid unit to determine input information and an output ratio, i.e., iₜ=v(xₜWₓᵢ+hₜ₋₁Wₕᵢ+bᵢ).

The input information is obtained through a tanh unit, i.e., Ĉₜ=tanh(xₜW_{xc}+hₜ₋₁W_{hc}+b_{c}).

The real input information after gating is iₜĈₜ, the cell information is jointly determined by the information left over from the last round and the information obtained at present, so cₜ=fₜcₜ₋₁+iₜ*Ĉₜ, i.e.,

cₜ=fₜcₜ₋₁+iₜ*tanh(xₜW_{xc}+hₜ₋₁W_{hc}+b_{c}).

The obtained cell information is processed via the tanh unit to obtain output information of the hidden, and at the same time, there is an output gate that controls an output through the sigmoid unit, including:
oₜ=σ(xₜWₓₒ+hₜ₋₁Wₕₒ+cₜ₋₁W_{co}+bₒ);
hₜ=oₜ tanh(cₜ);
and a final output ŷₜ is: ŷₜ=softmax (hₜ).

In the present disclosure, S2 includes the following steps.

S201. Historical blood glucose data of diabetic patients to be predicted are collected as a second database.

S202. The second database is imported.

Requirements for blood glucose data collection in S201 includes that a blood glucose testing instrument must collect for at least 4 days in 7 consecutive days, and at least 96 hours of continuous blood glucose data must be collected, in which at least 24 hours are spent overnight (i.e., from 10 pm to 6 am). As can be seen above that the second database includes a large amount of CGM data and lasts for a long time. For the same patient, there are sufficient data to study a blood glucose prediction algorithm for type 1 diabetes, which can make full use of the long-term (using historical data of 8 hours) and short-term (using historical data of 30 minutes) characteristics of the blood glucose data.

In this embodiment, samples in the second database are blood glucose data of patients with type 1 diabetes, and the age of the sample population ranges from 3.5 to 17.7 years old, with an average age of 9.9 years old.

In the present disclosure, S3 includes the following steps.

S301. Patient blood glucose data including missing values are processed by using a data missing value imputation method.

S302. The blood glucose data are smoothed by using a data smoothing filtering method.

The data missing value imputation method includes bilinear interpolation and linear extrapolation, and the data smoothing filtering method includes Kalman filtering and median filtering.

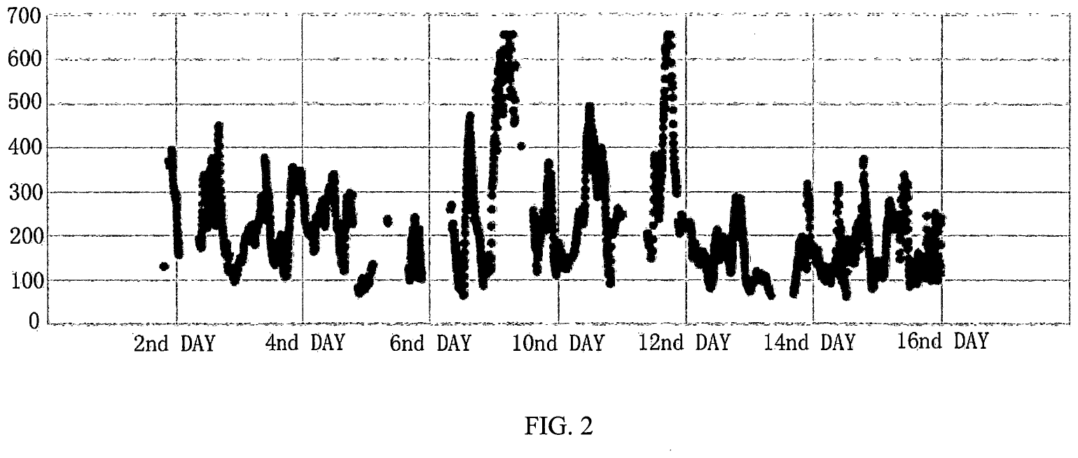

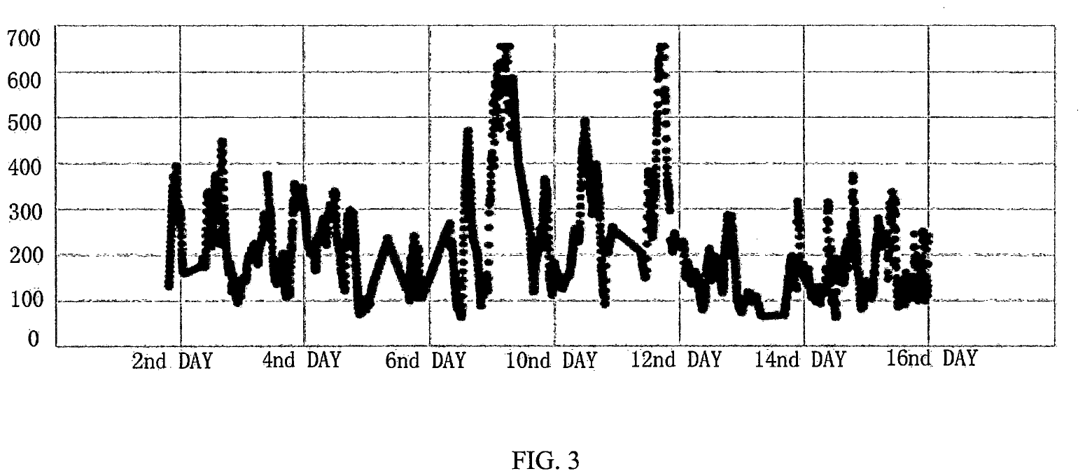

As shown in FIG. 2 and FIG. 3, the bilinear interpolation is a common method for reconstructing exact data points in two dimensions, which is suitable for the missing of known data, and will not be described in detail here. FIG. 2 and FIG. 3 show graphs of blood glucose concentration curves before and after using the bilinear interpolation, FIG. 2 shows a blood glucose concentration curve before bilinear interpolation processing, FIG. 3 shows a blood glucose concentration curve after bilinear interpolation processing, and the ordinate in the figure shows a blood glucose concentration of a patient in mg/dl. It can be seen that after using the bilinear interpolation, the missing values of the blood glucose data are imputed.

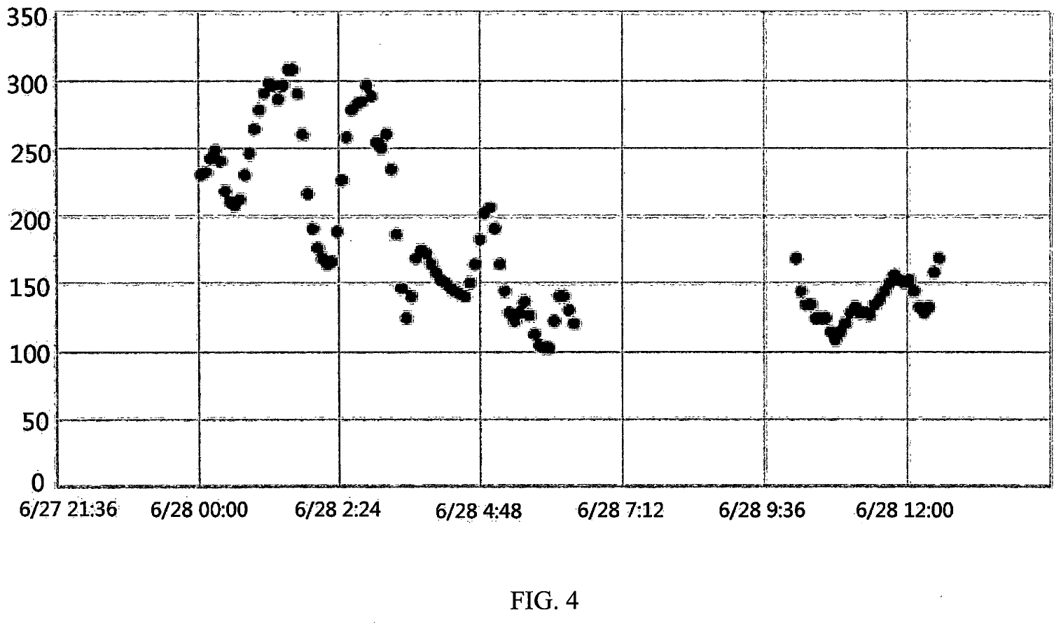

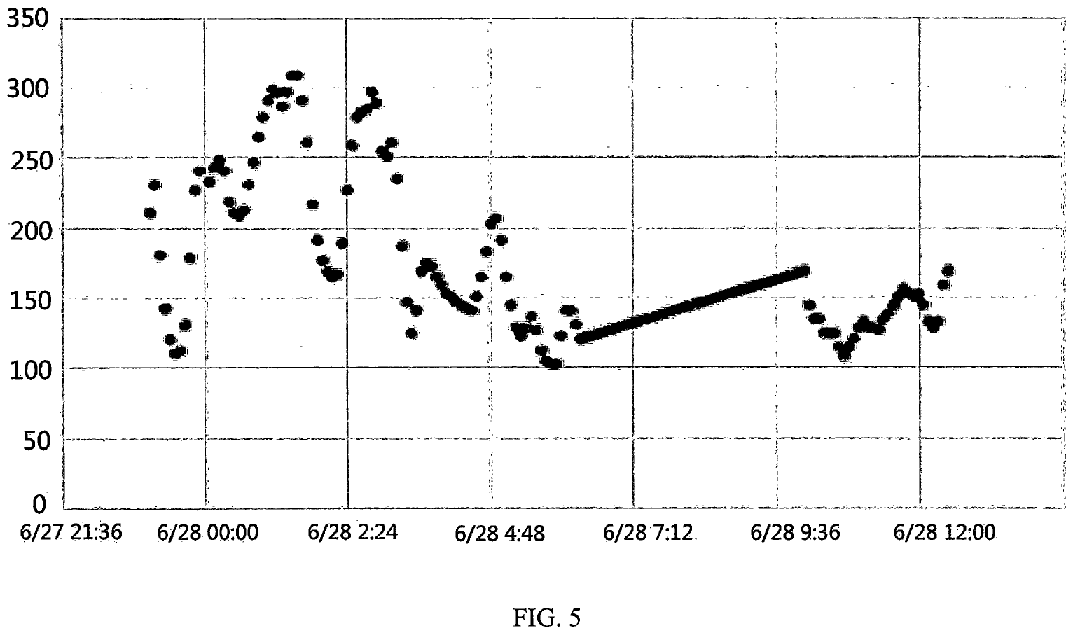

As shown in FIG. 4 and FIG. 5, the linear extrapolation is used to study things that change at a constant rate of growth over time. In a coordinate diagram with time as the abscissa, the change of things is close to a straight line. According to this straight line, the future change of things can be inferred, which is also one of the conventional data missing value imputation methods, and will not be described in detail here. FIG. 4 and FIG. 5 show graphs of blood glucose concentration curves before and after using the linear extrapolation. FIG. 4 shows a blood glucose concentration curve before linear extrapolation processing, FIG. 5 shows a blood glucose concentration curve after linear extrapolation processing. The ordinate in the figure shows a blood glucose concentration of a patient in mg/dl. It can be seen that after using the linear extrapolation method, the missing values of the blood glucose data are imputed.

The implementation steps of the Kalman filtering include prediction and correction. The prediction is to estimate a state of the current time based on a state estimation of the previous time, while the correction is to synthesize an estimated state and an observed state of the current time to estimate an optimal state.

The prediction and correction processes are as follows:

xₖ=Axₖ₋₁+BUₖ₋₁ (2-1);

Pₖ=APₖ₋₁A^{T}+Q (2-2);

Kₖ=PₖH^{T}(HPₖH^{T}+R)⁻¹ (2-3);

xₖ=xₖ+Kₖ(Zₖ−Hxₖ) (2-4);

Pₖ=(I−KₖH)Pₖ (2-5);
where Formula (2-1) is a state prediction, Formula (2-2) is an error matrix prediction, Formula (2-3) is a Kalman gain calculation, Formula (2-4) is a state correction, with an output being the final Kalman filtering result, and Formula (2-5) is an error matrix update.

The variables are described as follows: xₖ is a state of time K; A is a state transition matrix, which is related to a specific linear system; uₖ is the effect of the outside world on the system at the time K; B is an input control matrix, which is used to transform the external influence into the influence on the state; P is an error matrix; Q is a prediction noise covariance matrix; R is a measured noise covariance matrix; H is an observation matrix; Kₖ is a Kalman gain at the time K; zₖ is an observation value at the time K.

In the present disclosure, S4 includes the following steps.

S401. Historical blood glucose data of the past 1 hour, the past 3 hours, and the past 8 hours are chosen.

S402. A complete ensemble empirical mode decomposition with adaptive noise (CEEMDAN) is used to perform rolling decomposition on the chosen data, a time step of the rolling decomposition is set to be two days, so as to obtain signals with different frequencies, i.e., several IMF components.

2000 pieces of data are taken as an example to describe the rolling decomposition. Firstly, 1-576 pieces of blood glucose data are sent to the ensemble empirical mode decomposition model for decomposition; and then 2-577 pieces of blood glucose data are sent to the ensemble empirical mode decomposition model for decomposition, and so on.

CEEMDAN (Complete Ensemble Empirical Mode Decomposition with Adaptive Noise) is a complete ensemble empirical mode decomposition with adaptive noise, and at the same time, Gaussian noise is added to EEMD and noise is canceled by superposition and averaging for many times. EMMD is used to perform direct EMD decomposition on M signals added with white noise, and then to directly average the corresponding IMF. The CEEMDAN method is to add white noise (or IMF component of white noise) to the residual value every time one order of IMF component is obtained, then calculate the mean value of IMF components at this time, and iterate successively.

The algorithm principle involved in is as follows: assuming that Eᵢ (·) is an i-th intrinsic mode component obtained after EMD decomposition, the first intrinsic mode component obtained after CEEMDAN decomposition is C̅ₗ̅(̅t̅)̅, v^{j} is a Gaussian white noise signal satisfying a standard normal distribution, j=1, 2, . . . , N is the number of times of adding white noise, E is a standard table of white noise, and y(t) is a signal to be decomposed.

Specific decomposition steps are as follows.

(1) Gaussian white noise is added to a signal y(t) to be decomposed to obtain a new signal y(t)+(−1)^{q}εv^{j}(t), where q=1, 2, the new signal is subjected to EMD decomposition to obtain a first-order intrinsic mode component C₁:
E(y(t)+(−1)^{q}εv^{j}(t))=C₁^{j}(t)+r^{j}
in which, Eᵢ (·) is an i-th intrinsic mode component obtained after EMD decomposition, v¹ is a Gaussian white noise signal satisfying a standard normal distribution, j=1, 2, . . . , N is the number of times of adding white noise, E is a standard table of white noise, and y(t) is a signal to be decomposed.

(2) N generated mode components are subjected to overall average to obtain a first intrinsic mode component after CEEMDAN decomposition:

$$\overline{{C}_{1} ( t )} = \frac{1}{N} {\sum }_{J = 1}^{N} {C}_{1}^{j} ( t ) .$$

(3) A residual after removing the first mode component is calculated:
r₁(t)=y (t)−C̅₁̅(̅t̅)̅.

(4) Positive-negative paired Gaussian white noises are added into rᵢ(t) to obtain a new signal, and EMD decomposition is performed with the new signal as a carrier to obtain the first-order mode component D₁, thus obtaining a second intrinsic mode component after CEEMDAN decomposition:

$$\overline{{C}_{2} ( t )} = \frac{1}{N} {\sum }_{J = 1}^{N} {D}_{1}^{j} ( t ) .$$

(5) A residual after removing the second mode component is calculated:
r₂(t)=r₁(t)−C̅₂̅(̅t̅)̅.

(6) Above steps are repeated until the obtained residual signal is a monotone function and cannot be decomposed continuously, and the algorithm is end. If the number of intrinsic mode components obtained at this time is k, the original signal y(t) is decomposed into:
y(t)=Σₖ₌₁^{K}C̅ₖ̅(̅t̅)̅+rₖ(t).

The time sequence of the blood glucose concentration is a typical non-linear and non-stationary sequence due to its highly time-varying property, and the direct use of a recurrent neural network for the blood glucose sequence prediction may reduce the accuracy of prediction to some extent. By using the method for decomposing the complex blood glucose data, the nonlinear blood glucose data can be decomposed into subsequences with relatively single frequency components, and finally the historical data of a single patient can be decomposed into low-frequency approximate components (trend components or main components) and high-frequency detailed components (transient changes and noise components). Compared with a method for one-time complete sequence decomposition of blood glucose data, a data decomposition technology is used and transformed into the form of rolling decomposition. The rolling decomposition is used to decompose the blood glucose data in a certain period time, such as the past 1 hour, the past 3 hours, the past 8 hours, etc., such that the blood glucose fluctuation of the patient in a certain period of time can be observed better. Moreover, after using the rolling decomposition, the blood glucose data difference after each rolling prediction can be observed better.

In the present disclosure, S5 includes the following steps.

S501. The degree of chaos among the IMF components is calculated, and calculated entropy values are ranked according to results from large to small.

The subsequences with relatively single frequency components obtained after rolling decomposition can be subjected to entropy ranking recombination. Entropy values of the subsequences obtained after rolling decomposition are ranked from large to small by using a sample entropy and a permutation entropy, the sequence with a small entropy value is ranked first, and the sequence with a large entropy is ranked last. The subsequences obtained after rolling decomposition are recombined in order, such that the entropy values of the subsequences obtained after each rolling decomposition are ranked in the same order, i.e., the prediction effect of the model can be improved by ranking the ease of prediction of the sub-sequences obtained after each decomposition.

S502. The component with the maximum entropy value is subjected to secondary decomposition to maintain entropy values of all decomposed components within a certain interval, thus reducing the nonlinearity and non-stationarity of the blood glucose data.

When the component with the maximum entropy value is subjected to the secondary decomposition, a variational modal decomposition model (VMD) is adopted. VMD steps are as follows: Firstly, a variational problem is constructed. Assuming that an original signal f is decomposed into k components, it is guaranteed that a decomposition sequence is a mode component with a limited bandwidth having a central frequency, and the sum of estimated bandwidths of various modes is the smallest, and a constrained condition is that the sum of all modes is equal to the original signal, then a corresponding constrained variational expression is as follows:

$${min}_{\{ {μ}_{k} \} \{ {ω}_{k} \}} \{ {\sum }_{k} {‖ {\partial }_{t} [ ( δ ( t ) + \frac{j}{π t} * {u}_{k} ( t ) ] {e}^{- j {ω}_{k} t} ‖}_{2}^{2} \} \qquad ( 3 - 1 )$$

$$s . t .$$

$${\sum }_{k = 1}^{K} {u}_{{k}^{=}} f ;$$

where K is the number of modes to be decomposed (positive integer), {μₖ} {ωₖ} correspond to a k-th mode component and the central frequency after decomposition, δ₍ₜ₎ is a Dirac function, and * is a convolution operator.

The, above Formula (3-1) is solved, a Lagrange multiplication operator λ is introduced to transform the constrained variational problem into an unconstrained variational problem, thus obtaining an augmented Lagrange expression as follows:

$$ℒ ( \{ {u}_{k} \} , \{ {ω}_{k} \} , λ ) := α {\sum }_{k} {‖ {\partial }_{t} [ ( δ ( t ) + \frac{j}{π t} * {u}_{k} ( t ) ] {e}^{- j {ω}_{k} t} ‖}_{2}^{2} + {‖ f ( t ) - {\sum }_{k} {u}_{k} ( t ) ‖}_{2}^{2} + 〈 λ ( t ) , f ( t ) - {\sum }_{k} {u}_{k} ( t ) 〉 ; \qquad ( 3 - 2 )$$

in which, λ is the Lagrange multiplication operator and a is a quadratic penalty factor, which plays a role in reducing the interference of the Gaussian noise. By using alternating direction multiplier (ADMM) iterative algorithm combined with Parseval/Plancherel and Fourier isometric transformation, various mode components and center frequencies are optimized, and saddle points of augmented Lagrange function are searched. The expressions of post-uk, ωk and λ after alternating optimization iteration are as follows:

$${\hat{u}}_{k}^{n + 1} ( ω ) = \frac{\hat{f} ( ω ) - {\sum }_{i \neq k} {\hat{u}}_{i} ( ω ) + \hat{λ} ( ω ) / 2}{1 + 2 a {( ω - ω k )}^{2}} ; \qquad ( 3 - 3 )$$

$${ω}_{k}^{n + 1} = \frac{{\int }_{0}^{∞} ω {\vert {\hat{u}}_{k}^{n + 1} ( ω ) \vert}^{2} d ω}{{\int }_{0}^{∞} {\vert {\hat{u}}_{k}^{n + 1} ( ω ) \vert}^{2} d ω} ; \qquad ( 3 \text{-4)}$$

$${\hat{λ}}^{n + 1} ( ω ) = {\hat{λ}}^{n} ( ω ) + γ ( \hat{f} ( ω ) - {\sum }_{k} {\hat{u}}_{k}^{n + 1} ( ω ) ) ; \qquad ( 3 - 5 )$$

in which γ is a noise tolerance satisfying fidelity requirements of signal decomposition; ûₖⁿ⁺¹(ω), ûᵢ(ω), \hat{f}(ω), \hat{λ}(ω) correspond to Fourier Transformations of ûₖⁿ⁺¹(t), ûᵢ(t), \hat{f}(t) and \hat{λ}(t), respectively; \hat{λ}ⁿ(ω) is a n-th iteration of the Lagrange multiplication operator in a frequency domain; and uₖⁿ⁺¹(ω) is a (n+1)-th iteration of the mode μₖ in the frequency domain.

The main iterative solution process of the VMD is as follows.

S1. ûₖ¹, ωₖ¹, λ₁ and the maximum number of iterations N are initialized.

S2. ûₖ and ωₖ are updated by using Formula (3-3) and Formula (3-4).

S3. A is updated by using Formula (3-5).

S4. Accuracy convergence criterion E is greater than 0, in a case that Σₖ∥ûₖⁿ⁺¹−ûₖⁿ∥₂²/∥ûₖⁿ∥₂²<ε is not satisfied and N is less than N, the process returns to the second step, otherwise, the iteration is completed, and the final ûₖ and ωₖ are output.

In the present disclosure, the ensemble learning module in S6 includes multiple different machine learning algorithms, and importing the data of the diabetic patients processed in step S5 specifically includes the following steps.

S601. The data are sent to three different machine learning algorithms, e.g., LSTM, GRU and SRNN, so as to obtain multiple prediction results.

S602. The multiple prediction results are combined as a basic prediction result.

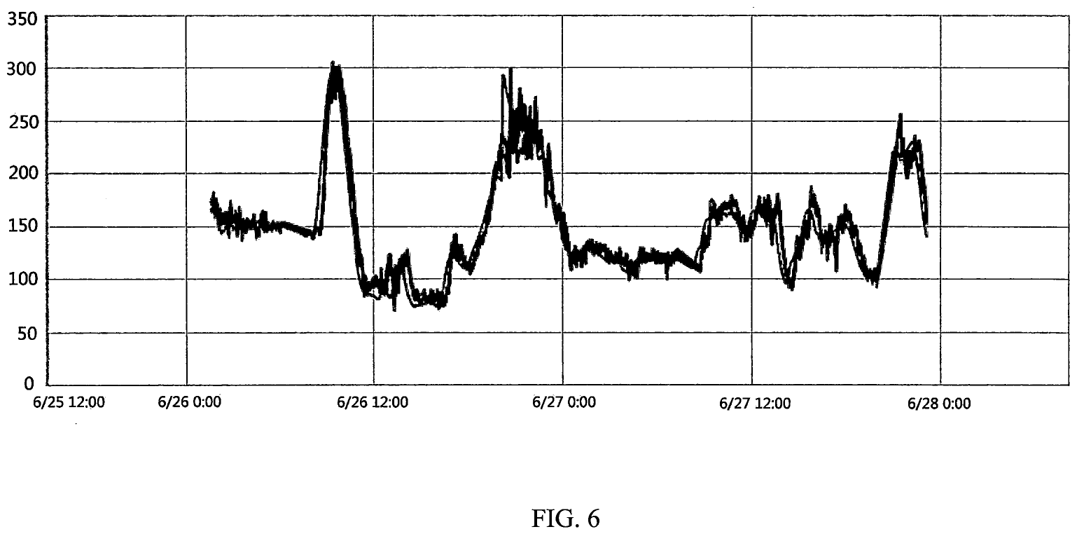

S603. The basic prediction result obtained in S602 is as a training set, and the training set is sent into a model Nested-LSTM to obtain the final prediction result. Referring to FIG. 6, the ordinate denotes the blood glucose concentration of the patient, which is expressed in mg/dl.

As can be seen above that in the application of ensemble learning, instead of using a single machine learning algorithm, the learning task is completed by constructing and combining multiple machine learners. Different network models can learn and combine the corresponding blood glucose characteristics, so as to achieve a better blood glucose prediction effect.

The above Nested-LSTM model (long and short-time memory network) is improved from the LSTM model, the learned function Cₜ=mₜ(fₜ⊙Cₜ₋₁, iₜ⊙gₜ) is used to replace an add operation for calculating C_{C} in the LSTM. A state of the function is expressed as internal memory of m at time t, and the function is called to calculate C_{C} and mₜ₊₁. Another LSTM unit is used to achieve the memory function and generate the Nested-LSTM model. When the memory function is replaced with another Nested-LSTM unit, an arbitrarily-deep nested network is constructed. The input and hidden states of the memory function in the Nested-LSTM are as follows:
\tilde{h}ₜ₋₁=fₜ⊙cₜ₋₁;
\tilde{x}ₜ=iₜ⊙σ_{c}(xₜW_{xc}+hₜ₋₁W_{hc}+b_{c}).

When the memory function is additive, the state of the memory cell is updated to:
Cₜ=hₜ₋₁+\tilde{x}ₜ.

An operation mode of the internal LSTM model is controlled by the following set of equations:

ĩₜ=\tilde{σ}ₜ(\tilde{x}ₜ\tilde{W}ₓᵢ+\tilde{h}ₜ₋₁\tilde{W}ₕᵢ+\tilde{b}ᵢ),

\tilde{f}ₜ=\tilde{σ}_{f}(\tilde{x}ₜ\tilde{W}_{xf}+\tilde{h}ₜ₋₁\tilde{W}_{hf}+\tilde{b}_{f}),

\tilde{c}ₜ=\tilde{f}ₜ⊙\tilde{c}ₜ₋₁+ĩₜ⊙\tilde{σ}ₜ(\tilde{x}ₜ\tilde{W}_{xc}+\tilde{h}ₜ₋₁\tilde{W}_{hc}+\tilde{b}_{c}),

õₜ=\tilde{σ}ₒ(\tilde{x}ₜ\tilde{W}ₓₒ+\tilde{h}ₜ₋₁\tilde{W}ₕₒ+\tilde{b}ₒ),

\tilde{h}ₜ=õₜ⊙\tilde{σ}ₕ(\tilde{c}ₜ).

Now, the unit state of the external LSTM is updated to:

cₜ=\tilde{h}ₜ.

To sum up, by using the pre-training model of transfer learning, the data after collecting is subjected to missing value imputation and smoothing filtering processing, and the data is processed by using a rolling data decomposition method, and the blood glucose concentration is predicted by using ensemble learning. Specifically, according to the blood glucose variation law of diabetic patients and the healthy people, the blood glucose values of the next 30 minutes and the next 1 hour can be predicted by using the data of the past 30 minutes, the past 1 hour, the past 2 hours, the past 4 hours and the past 8 hours. The constructed data set is pre-trained with machine learning algorithms, such as a GRU, a SRNN, an LSTM and other recurrent neural networks, and the trained model is used as a pre-training model for the subsequent task, that is, the parameters of the pre-training model should be loaded first when training the subsequent model; the historical blood glucose data of the patients to be predicted are subjected to data processing (missing imputation processing and smoothing processing) to make the overall data closer to the real blood glucose data; after the data processing is completed, the processed patient data is subjected to mode decomposition, such as CEEMDAN (Complete Ensemble Empirical Mode Decomposition with Adaptive Noise) technology; the processed blood glucose data is decomposed into a series of intrinsic mode components (IMF) with different frequency information, and the mode components obtained after the mode decomposition is subjected to sample entropy analysis, the component with the maximum sample entropy is subjected to secondary decomposition by using a variational mode decomposition (VMD) to significantly reducing the nonlinearity and instability of the blood glucose data. After obtaining the pre-training model and processing the data, the processed data are sent to the machine learning model for ensemble learning, and on this basis, the two-stage prediction method is adopted to further improve the prediction effect of the model, and then to obtain the final prediction result.

The technical features of above embodiments may be arbitrarily combined, and all possible combinations of the technical features in the above-described embodiments are not described for simplicity of description. However as long as the combinations of technical features do not contradict each other, the technical features should be considered to be within scope of description of present specification.

The above embodiments only express some implementations of the present disclosure, and the descriptions thereof are relatively specific and detailed, but cannot be understood as limiting the scope of patent protection of the present disclosure. It should be noted that various modifications and improvements that can be made by those of ordinary skill in the art without departing from the concept of the present disclosure belong to the scope of protection of the present disclosure. Therefore, the scope of patent protection of the present disclosure shall be on the basis of the appended claims.

What is claimed is:

1. A two-stage blood glucose prediction method based on pre-training and data decomposition, comprising the following steps:
S1, combining blood glucose data of healthy people and diabetic people to develop a pre-training model;
S2, collecting data of diabetic patients to be predicted;
S3, performing missing value imputation processing and smooth processing on the data obtained in S2;
S4, performing mode decomposition on the data obtained in S3 to obtain intrinsic mode components with different frequency information;
S5, performing sample entropy analysis on the mode components obtained by decomposing in S4, and performing secondary decomposition on a component with the maximum sample entropy; and
S6, loading a weight of the pre-training model obtained in S1, and importing the data of the diabetic patients processed in S5 into an ensemble learning module, wherein the ensemble learning module is used for predicting blood glucose values in the next 30 minutes and the next 60 minutes.

2. The two-stage blood glucose prediction method based on pre-training and data decomposition according to claim 1, wherein S1 comprises the following steps:
S101, importing a first database, wherein samples in the first database comprise the blood glucose data of the diabetic people and the blood glucose data of the healthy people;
S102, screening out historical blood glucose data of the past 30 minutes, the past 1 hour, the past 2 hours, the past 4 hours and the past 8 hours; and
S103, sending the screened blood glucose data to an LSTM model, saving training results as a weight file, wherein the weight file is used as a pre-training model and as default parameters of a subsequent training model.

3. The two-stage blood glucose prediction method based on pre-training and data decomposition according to claim 2, wherein the blood glucose data in S101 is continuous blood glucose monitoring data of 50 consecutive days; and sample population comprise a plurality of children, a plurality of adolescents, and a plurality of adults.

4. The two-stage blood glucose prediction method based on pre-training and data decomposition according to claim 1, wherein S2 comprises the following steps:
S201, collecting historical blood glucose data of the diabetic patients to be predicted as a second database; and
S202, importing the second database.

5. The two-stage blood glucose prediction method based on pre-training and data decomposition according to claim 4, wherein requirements for blood glucose data collection in S201 comprise that a blood glucose testing instrument collects for at least 4 days in 7 consecutive days, and that at least 96 hours of continuous blood glucose data needs to be collected.

6. The two-stage blood glucose prediction method based on pre-training and data decomposition according to claim 1, wherein S3 comprises the following steps:
S301, processing patient blood glucose data including missing values using a data missing value imputation method; and
S302, smoothing the blood glucose data using a data smoothing filtering method.

7. The two-stage blood glucose prediction method based on pre-training and data
decomposition according to claim 1, wherein the data missing value imputation method comprises bilinear interpolation and linear extrapolation, and the data smoothing filtering method comprises Kalman filtering and median filtering.

8. The two-stage blood glucose prediction method based on pre-training and data decomposition according to claim 1, wherein S4 comprises the following steps:
S401, choosing historical blood glucose data of the past 1 hour, the past 3 hours, and the past 8 hours; and
S402, performing rolling decomposition on the chosen data by using an ensemble empirical mode decomposition model, wherein a time step of the rolling decomposition is set to be two days, so as to obtain signals with different frequencies, that is, a plurality of IMF components.

9. The two-stage blood glucose prediction method based on pre-training and data decomposition according to claim 1, wherein S5 comprises the following steps:
S501, calculating the degree of chaos among the IMF components, and ranking calculated entropy values according to results from large to small; and
S502, performing secondary decomposition on a component with the maximum entropy value to maintain entropy values of all decomposed components within a certain interval, and reducing nonlinearity and non-stationarity of the blood glucose data.

10. The two-stage blood glucose prediction method based on pre-training and data decomposition according to claim 1, wherein the ensemble learning module in S6 comprises a plurality of different machine learning algorithms, and importing the data of the diabetic patient processed in S5 specifically comprises the following steps:
S601, sending the data to three different machine learning algorithms at first: an LSTM, a GRU and a SRNN, so as to obtain a plurality of prediction results;
S602, combining the plurality of prediction results as a basic prediction result;
S603, serving the basic prediction result obtained in step S602 as a training set, and sending the training set to a model Nested-LSTM to obtain a final prediction result.
